(19) United States
(12) Plant Patent Application Publication
(10) Pub. No.: US 2014/0047597 P1
(43) Pub. Date: Feb. 13, 2014

(54) Strawberry plant named 'ZAFIR'

(50) ;

(71) Applicant: C.I.V.- CONSORZIO ITALIANO VIVAISTI - SOCIETÁ CONSORTILE A R.L, (US)

(72) Inventors: Michelangelo Leis, Ferrara (IT); Alessio Martinelli, Ferrara (IT); Gianfranco Castagnoli, Quingentole (IT)

(73) Assignee: C.I.V.- CONSORZIO ITALIANO VIVAISTI - SOCIETÁ CONSORTILE A R.L, Comacchio FE (IT)

(21) Appl. No.: 13/694,851

(22) Filed: Jan. 11, 2013

(30) Foreign Application Priority Data
Aug. 9, 2012 (QZ) 2012/1734

(51) Int. Cl. A01H 5/00

(52) U.S. Cl. PLT/208

(57) ABSTRACT

A new and distinct variety of Fragaria L. named ‘ZAFIR’, characterized by the description herein.

### LATIN NAME OF THE GENUS AND SPECIES CLAIMED

Fragaria L.

### VARIETY DENOMINATION

‘ZAFIR’

### CROSS REFERENCE TO RELATED APPLICATIONS

This application claims priority under 35 U.S.C. §119(f) of the Community plant variety office (CPVO) application No. 2012/1734 filed Aug. 9, 2012

### BACKGROUND OF THE INVENTION

The present invention relates to a new and distinct strawberry variety named ‘ZAFIR’, which was the result of a controlled cross performed in 2004 between the strawberry variety ‘VENTANA’ (Patented, U.S. Plant Pat. No. 13,469) and an proprietary unpatented selection of Consorzio Italiano Vivaisti named ‘R6R1-26’.

The progeny was first asexually propagated by stolons in S. Giuseppe di Comacchio Ferrara, Italy, in 2005.

The ‘ZAFIR’ variety was tested over the next several years in different European areas with low chill conditions. The tests started in 2006 and continued until 2011.

### BRIEF SUMMARY OF THE INVENTION

Plants of the new Strawberry variety ‘ZAFIR’ can be distinguished from plants of the comparison Strawberry variety ‘CIVRI-30’ (Registered, CPVO Grant No. 14561) by the following unique combination of characteristics described in Table 1:

TABLE 1
Comparison with Comparison Variety

| Denomination | Characteristic in | State of | State of |
|---|---|---|---|
| of similar | which the similar | expression of | expression of |
| variety | variety is different | similar variety | candidate variety |
| CIVRI-30 | Color | Orange Red | Medium Red |
| | Firmness | Medium | Firm |

Asexual reproduction of this new variety by stolons shows that the foregoing and all other characteristics and distinctions come true to form and are established and transmitted through succeeding propagations. The new variety reproduces true to type.

### BRIEF DESCRIPTION OF THE PHOTOGRAPHS

The accompanying photographs illustrate the overall appearance of the new Strawberry ‘ZAFIR’ showing the colors as true as is reasonably possible with colored reproductions of this type. Colors in the photographs may differ slightly from the color value cited in the detailed botanical description, which accurately describe the color of ‘ZAFIR’.

FIG. 1 shows the plant of ‘ZAFIR’;

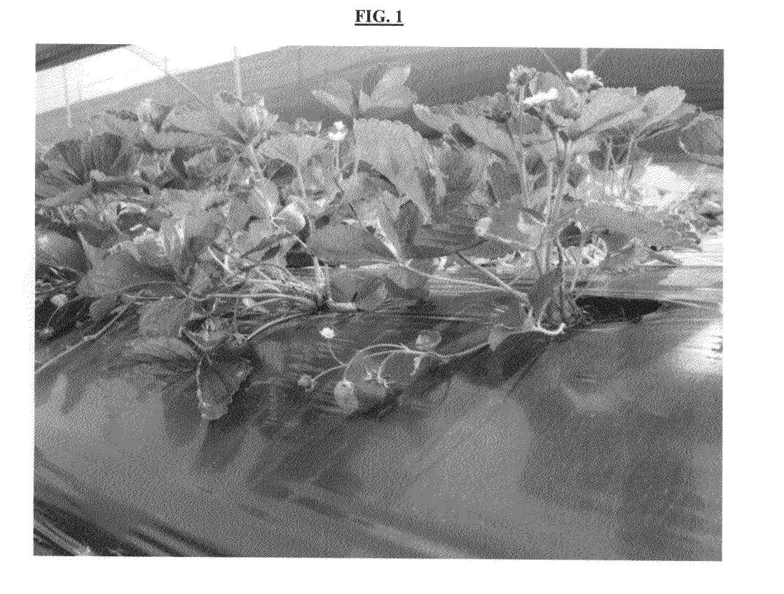

FIG. 2 shows the leaves of ‘ZAFIR’;

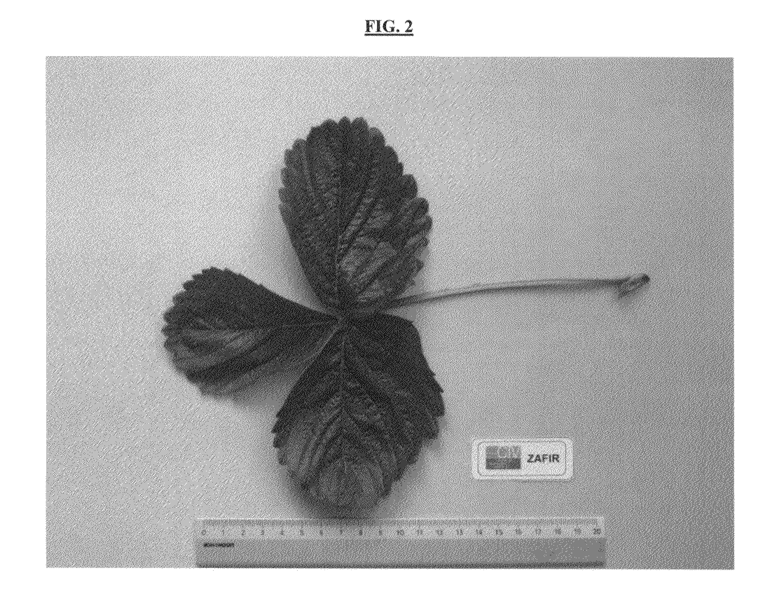

FIG. 3 shows the flowers of ‘ZAFIR’; and

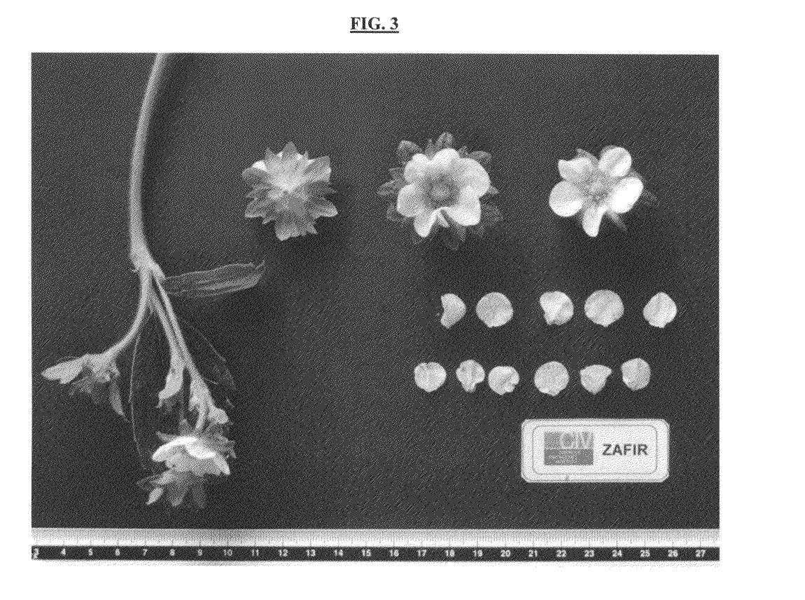

FIG. 4 shows the fruits of ‘ZAFIR’.

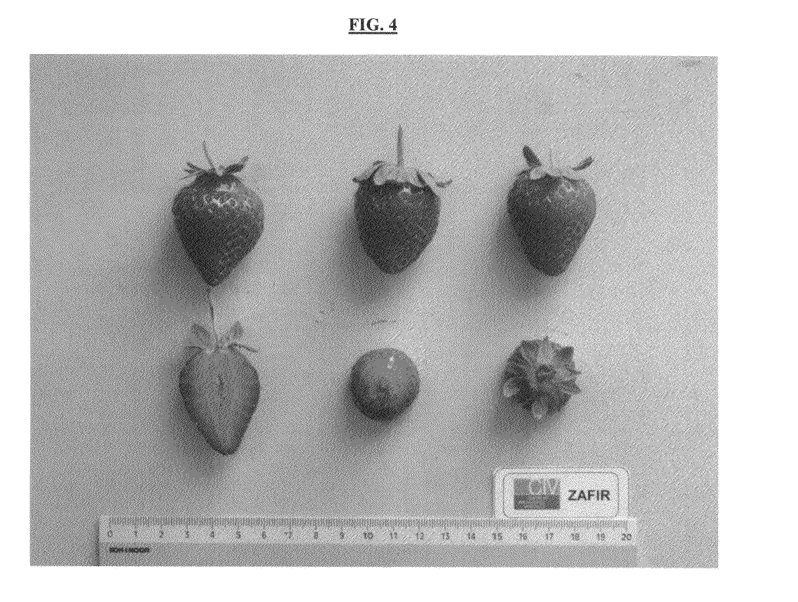

The colors of this illustration may vary with lighting conditions and, therefore, color characteristics of this new variety should be determined with reference to the observations described herein, rather than from these illustrations alone.

### DETAILED BOTANICAL DESCRIPTION

The following detailed description of the ‘ZAFIR’ variety, unless otherwise noted, is based on observations taken of plants and fruits, and asexually reproduced progeny, grown at S. Giuseppe di Comacchio, Ferrara, Italy.

The observed plants were one year old plants, produced in a nursery in S. Giuseppe di Comacchio, Italy, dug in December, cold stored, and then planted in production fields in the middle of April.

Certain characteristics of this variety may change with changing environmental conditions (e.g., light, temperature, moisture, etc.), nutrient availability, or other factors. Quantified measurements are expressed as an average of measurements taken from a number of individual plants of the new variety. The measurements of any individual plant, or any group of plants, of the new variety may vary from the stated average. Color descriptions and other terminology are used in accordance with their ordinary dictionary descriptions, unless the context clearly indicates otherwise. Color designations are made with reference to The Royal Horticultural Society (R.H.S.) Color Chart.
Plant:
   Habit.—Semi-upright or spreading.
   Density.—Medium.
   Vigor.—Medium.
   Overall shape.—Slightly Upright.
   Height.—0.28 to 0.30 meters.
   Width.—About 0.40 meters.
Leaf:
   Color.—Upperside: RHS 137A green group; underside: RHS 137D green group.
   Size.—About 22 cm.
   Cross section.—Concave.
   Leaf surface undulation or blistering.—Medium.
   Number of leaflets.—Three only.
   Leaf stem characteristics.—Color: RHS 144A yellow-green group. Position of hairs: horizontal. Length: About 16 cm.
   Terminal leaflet.—Length/width ratio: Equal or moderately longer. Length: About 10.5 cm. Width: About 9.5 cm. Shape of base: Acute. Shape of teeth: Serrate to crenate.
   Petiole.—Position of hairs: Horizontal. Length: About 15-16 cm.
   Stipule.—Color RHS 145A yellow-green group; Anthocyanin coloration: absent to very weak.
   Stolons.—Number about 7-8. Thickness: Medium, about 3.3 mm. Pubescence: Medium. Color RHS 144B, yellow green with weak anthocyanin coloration, RHS 185D greyed-purple.
Inflorescence: Position relative to foliage: Beneath.
   Flower.—Size: Medium. Size of calyx relative to corolla: Larger. Spacing of petals. Free.
   Flower characteristics.—Diameter primary flowers. About 4 cm. Diameter secondary flowers. About 2.5-3 cm. Number of petals: normally about 5 to 6. Fragrance: No significant fragrance. Time from bloom to mature fruit: About 30 days.
Reproductive organs:
   Stamens.—Numerous with pollen present, fertile and abundant. Length: approximately 2 mm. Color: near RHS 145C, yellow-green.
   Anthers.—Size: Generally average, length: 1.3 mm; width: 0.9 mm. Color: RHS 14C yellow orange; darkening with advanced maturity.
   Pollen.—Fertile and abundant. Color: RHS 14A yellow orange.
   Pistils.—Numerous, generally average in size. Color: RHS 151C yellow green.
   Petal.—Length/width ratio: equal.
Fruit:
   Fruit truss.—Attitude: Semi-erect.
   Ratio of length/maximum width.—moderately longer than broad.
   Color of mature fruit.—RHS 46A, red.
   Color of internal fruit.—RHS 44B orange-red.
   Color of core.—RHS 33A orange-red.
   Length.—32-38 mm.
   Width.—25-32 mm.
   Calyx diameter.—26-33 mm.
   Average weight.—15-20 grams.
   Achene color.—RHS 153B yellow-green.
   Number of achenes per cm².—about 8.0.
   Achene size.—length about 1.4 mm, width about 1.0 mm.
   Weight of 1000 achenes.—0.70 grams.
   Peduncle length of inflorescence stem.—Primary fruit 2.5 to 3.5 cm, secondary fruit about 2.0 cm, color near RHS 145A, yellow-green.
   Marketable yield (gm/plant).—about 800-1000 grams.
   Fruit size.—large.
   Predominant shape.—conical-ovoid.
   Difference in shapes between primary and secondary fruit.—slight.
   Band without achenes.—absent to very narrow.
   Unevenness of surface.—even to very slightly uneven.
   Evenness of color.—even to very slightly uneven.
   Glossiness.—medium.
   Insertion of achenes.—below surface.
   Insertion of calyx.—level with fruit.
   Attitude of the calyx.—outwards.
   Size of calyx in relation to fruit diameter.—same size to slightly larger.
   Adherence of calyx.—medium.
   Firmness of flesh.—firm.
   Hollow center expression.—Small.
   Flavor.—good.
   Soluble solids (% brix).—average 7.5°.
   Time of first flowering.—about 30 days after planting using cold stored plants quality A.
   Time of first harvesting.—about 30-35 days after first flowering.
   Harvest period.—June to November.
   Type of bearing.—fully remontant.
Disease resistance: The ‘ZAFIR’ variety has been tested for several years and it has manifest good vigor and good tolerance to diseases. No specific resistance assessment has been done.

What is claimed is:

1. A new and distinct variety of Fragaria L. named ‘ZAFIR’, as illustrated and described herein.
